(19) United States
(12) Patent Application Publication
(10) Pub. No.: US 2008/0161583 A1
(43) Pub. Date: Jul. 3, 2008

(54) Epimerization methodologies for recovering stereoisomers in high yield and purity

(76) Inventors: Gillian M. Nicholas, Boulder, CO (US); Robert J. Topping, Longmont, CO (US)

Correspondence Address: ROCHE PALO ALTO LLC;Patent Law Department, M/S A2-250, 3431 Hillview Avenue, Palo Alto, CA, 94304, US

(21) Appl. No.: 12/004,266

(22) Filed: Dec. 20, 2007

Related U.S. Application Data
(60) Provisional application No. 60/877,877, filed on Dec. 29, 2006.

(51) Int. Cl. C07D 319/08

(52) U.S. Cl. 549/333

(57) ABSTRACT

The present invention relates to methods of subjecting a mixture of stereoisomers to epimerization and optionally recrystallization procedures to obtain a desired stereoisomer in high yield and purity. Relying upon solubility differences, the epimerization desirably is carried out in a solvent mixture that extends the epimerization equilibrium in favor of the desired stereoisomer. Recrystallization from a solvent mixture upgrades the purity even more. Purified stereoisomers are useful in many applications such as intermediates in the synthesis of pharmacologically important molecules.

### PRIORITY CLAIM

The present non-provisional patent Application claims benefit from U.S. Provisional Patent Application Ser. No. 60/877,877, filed on Dec. 29, 2006, by Topping et al. and titled EPIMERIZATION METHODOLOGIES FOR RECOVERING STEREOISOMERS In HIGH YIELD AND PURITY, wherein the entirety of said provisional patent application is incorporated herein by reference.

### FIELD OF THE INVENTION

The present invention relates to methods of subjecting a mixture of stereoisomers to epimerization and recrystallization procedures to obtain a desired stereoisomer in high yield and purity. Relying upon solubility differences, the epimerization desirably is carried out in a solvent mixture that extends the epimerization equilibrium in favor of the desired stereoisomer. Recrystallization from a solvent mixture further upgrades the purity. Purified stereoisomers are useful in many applications such as intermediates in the synthesis of pharmacologically important molecules.

### BACKGROUND OF THE INVENTION

Glucokinase (GK) is one of four hexokinases that are found in mammals [Colowick, S. P., in The Enzymes, Vol. 9 (P. Boyer, ed.) Academic Press, New York, N.Y., pages 1-48, 1973]. The hexokinases catalyze the first step in the metabolism of glucose, i.e., the conversion of glucose to glucose-6-phosphate. Glucokinase has a limited cellular distribution, being found principally in pancreatic β-cells and liver parenchymal cells. In addition, GK is a rate-controlling enzyme for glucose metabolism in these two cell types that are known to play critical roles in whole-body glucose homeostasis [Chipkin, S. R., Kelly, K. L., and Ruderman, N. B. in Joslin's Diabetes (C. R. Khan and G. C. Wier, eds.), Lea and Febiger, Philadelphia, Pa., pages 97-115, 1994]. The concentration of glucose at which GK demonstrates half-maximal activity is approximately 8 mM. The other three hexokinases are saturated with glucose at much lower concentrations (<1 mM).

Therefore, the flux of glucose through the GK pathway rises as the concentration of glucose in the blood increases from fasting (5 mM) to postprandial (≈10-15 mM) levels following a carbohydrate-containing meal [Printz, R. G., Magnuson, M. A., and Granner, D. K. in Ann. Rev. Nutrition Vol. 13 (R. E. Olson, D. M. Bier, and D. B. McCormick, eds.), Annual Review, Inc., Palo Alto, Calif., pages 463-496, 1993]. These findings contributed over a decade ago to the hypothesis that GK functions as a glucose sensor in β-cells and hepatocytes (Meglasson, M. D. and Matschinsky, F. M. Amer. J. Physiol. 246, E 1-E 13, 1984).

In recent years, studies in transgenic animals have confirmed that GK does indeed play a critical role in whole-body glucose homeostasis. Animals that do not express GK die within days of birth with severe diabetes while animals overexpressing GK have improved glucose tolerance (Grupe, A., Hultgren, B., Ryan, A. et al., Cell 83, 69-78, 1995; Ferrie, T., Riu, E., Bosch, F. et al., FASEB J., 10, 1213-1218, 1996). An increase in glucose exposure is coupled through GK in β-cells to increased insulin secretion and in hepatocytes to increased glycogen deposition and perhaps decreased glucose production.

The finding that type II maturity-onset diabetes of the young (MODY-2) is caused by loss of function mutations in the GK gene suggests that GK also functions as a glucose sensor in humans (Liang, Y., Kesavan, P., Wang, L. et al., Biochem. J. 309, 167-173, 1995). Additional evidence supporting an important role for GK in the regulation of glucose metabolism in humans was provided by the identification of patients that express a mutant form of GK with increased enzymatic activity. These patients exhibit a fasting hypoglycemia associated with an inappropriately elevated level of plasma insulin (Glaser, B., Kesavan, P., Heyman, M. et al., New England J. Med. 338, 226-230, 1998). While mutations of the GK gene are not found in the majority of patients with type II diabetes, compounds that activate GK, and thereby increase the sensitivity of the GK sensor system, would still be useful in the treatment of the hyperglycemia characteristic of all type II diabetes. Glucokinase activators would increase the flux of glucose metabolism in β-cells and hepatocytes, which would be coupled to increased insulin secretion. Such agents would be useful for treating type II diabetes.

The following glucokinase activator (referred to herein as the compound of Formula I)

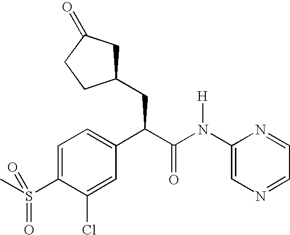

and its isopropanol (IPA) solvate of the formula:

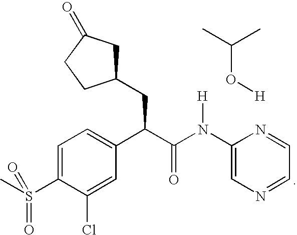

are under evaluation as a potentially new therapy for the treatment of Type 2 diabetes. The compound of Formula I has also been described in PCT Patent Publication No. WO 03/095438 as well as in the co-pending U.S. application Ser. No. 11/583,971, corresponding to U.S. Publication No. 2007/0129554, titled ALPHA FUNCTIONALIZATION OF CYCLIC, KETALIZED KETONES AND PRODUCTS THEREFROM, bearing Attorney Docket No. RCCO0021/US, and filed Oct. 19, 2006, in the names of Harrington et al (hereinafter Application A); and U.S. Provisional Application No. 60/791,256, titled PROCESS FOR THE PREPARATION OF A GLUCOKINASE ACTIVATOR, bearing Attorney Docket No. 23026, and filed Apr. 12, 2006 in the names of Andrzej Robert Daniewski et al., (hereinafter Application B); and U.S. Provisional Patent Application No. 60/877,878, titled REDUCTION MOTHODOLOGIES FOR CONVERTING KETAL ACIDS, SALTS AND ESTERS TO KETAL ALCOHOLS, bearing Attorney Docket No. RCCO0031/P1, filed Dec. 29, 2006, in the name of Robert J. Topping (hereinafter Application C); and U.S. Provisional Patent Application No. 60/877,788, titled AROMATIC SULFONATED KETALS, bearing Attorney Docket No. RCC0032/P1, filed Dec. 29, 2006 in the names of Robert J. Topping et al., (hereinafter Application D). All of these patent documents are incorporated herein by reference in their respective entireties for all purposes.

Application B schematically shows and describes a multi-step reaction scheme in which the compound of Formula I is manufactured from a ketal acid starting material in nine main reaction steps. In step 6 of this scheme, the reactant is a mixture of epimers substituted aromatic ketal acid (identified there as sulfone 7) that includes substantial amounts of the 2R,3′R and 2S,3′R stereoisomers, or epimers. The other 2R,3′S and 2S,3′S epimers exist only in de minimus amounts. Step 6 of that reaction scheme subjects the racemic material to an epimerization reaction in order to convert as much of the undesired 2S,3′R epimer to the desired 2R,3′R form as possible. The purified 2R,3′R reaction product (shown there as sulfone 8) is then converted to the compound of Formula I according to steps 7, 8 and 9.

In Step 6 of Application B, a mixture of the epimers is treated with a base such as sodium alkoxide in a solvent such as ethanol with heating. The undesired 2S,3′R epimer is much more soluble in ethanol than is the desired 2R,3′R epimer, which has a more limited solubility in the ethanol. Consequently, the 2R,3′R epimer tends to preferentially crystallize out of solution. The other epimer remains in the solution where it is converted to the desired epimer, which again preferentially crystallizes out of solution. The reaction proceeds in this manner until an equilibrium is reached. Beyond this point, further conversion of the undesired epimer to the other is accompanied by the creation of too great an amount of undesirable by-products such as aryl ethoxy and/or des-chloro impurities. Unfortunately, this equilibrium is reached when only about 84% of the epimer material is in the desired 2R,3′R form. It would be very desirable to conduct an epimerization that yields a greater percentage of the desired epimer without undue production of by-products.

Additionally, it is also more difficult than might be desired to recover and isolate the crystallized product resulting when step 6 of Application B is carried out. In particular, the desired epimer tends to crystallize from ethanol to produce crystals having poor filtration properties. This makes it difficult to use filtration to recover the crystals from the epimerization liquor. It would be very desirable to conduct an epimerization that yields the desired epimer crystals in a form that is more compatible with filtering techniques.

### SUMMARY OF THE INVENTION

The present invention relates to methods of subjecting a mixture of stereoisomers to epimerization and optionally recrystallization procedures to obtain a desired epimer in high yield and purity. Relying upon solubility differences, the epimerization desirably is carried out in a solvent mixture that extends the epimerization equilibrium in favor of the desired epimer. Recrystallization from a solvent mixture further upgrades the purity. Recrystallization also may improve the filtering characteristics of the product. Purified epimers are useful in many applications such as intermediates in the synthesis of pharmacologically important molecules.

In one aspect, the present invention relates to a method of epimerization. A mixture comprising first and second epimers is provided. The mixture is epimerized to convert at least a portion of the second isomer to the first epimer. The epimerization provides an epimerized product with an enriched content of the first epimer. Epimerization occurs in a basic reaction medium comprising first and second solvents; wherein:

the second epimer has greater solubility in the first and second solvents than does the first epimer, and

the first epimer is substantially insoluble in the second solvent.

### BRIEF DESCRIPTION OF THE DRAWINGS

The above mentioned and other advantages of the present invention, and the manner of attaining them, will become more apparent and the invention itself will be better understood by reference to the following description of the embodiments of the invention taken in conjunction with the accompanying drawings, wherein:

FIG. 1 schematically shows one reaction scheme for making a mixture of aromatic ketal acid epimers that proceeds through mesylate and iodide intermediates; and

FIG. 2 schematically shows one reaction scheme for making a mixture of aromatic ketal acid epimers that proceeds through a tosylate intermediate.

### DETAILED DESCRIPTION OF PRESENTLY PREFERRED EMBODIMENTS

The embodiments of the present invention described below are not intended to be exhaustive or to limit the invention to the precise forms disclosed in the following detailed description. Rather the embodiments are chosen and described so that others skilled in the art may appreciate and understand the principles and practices of the present invention.

The epimerization and resolution methodology of the present invention is beneficially applied to any stereoisomers having different solubility characteristics in different solvents. The methodology can be used to form, resolve and recover a desired stereoisomer with enhanced yield and purity. The methodology of the present invention will be described in the illustrative context of stereoisomers (hereinafter referred to as aromatic, ketal acids) that are organic acids, or salts thereof, that include a carboxylic acid moiety or salt thereof; a chiral carbon atom that has an H substituent and that is in an alpha position relative to the carboxylic acid (or salt) moiety; a ketal moiety that is linked to the chiral carbon atom by a suitable linking group; an aromatic moiety that is linked by a single bond or a linking group to the chiral carbon atom that is in an alpha position relative to the carboxylic acid (or salt) moiety; and an optional oxo-hetero substituent incorporated into the aromatic moiety.

The carboxylic acid (or salt) moiety generally has the formula —C(O)OM, wherein M is hydrogen or a suitable cation such as sodium, potassium, lithium, ammonium, combinations of these, and the like. Commonly, M is H or Na.

The ketal moiety is either pendant from, or constitutes at least a portion of the backbone, of the ketal linking group. A ketal moiety is a functional group that includes a carbon atom bonded to both —OZ¹ and —OZ² groups, wherein each of Z¹ and Z² independently may be a wide variety of monovalent moieties or co-members of a ring structure. In representative embodiments, Z¹ and Z² alone or as co-members of a ring structure are linear, branched, or cyclic alkyl(ene); preferably alkyl(ene) of 1 to 15, preferably 2 to 5 carbon atoms. The divalent, branched alkylene backbone associated with neopentyl glycol is a preferred structure when Z¹ and Z² are co-members of a ring structure.

A ketal is structurally equivalent to an acetal, and sometimes the terms are used interchangeably. In some uses, a difference between an acetal and a ketal derives from the reaction that created the group. Acetals traditionally derive from the reaction of an aldehyde and excess alcohol, whereas ketals traditionally derive from the reaction of a ketone with excess alcohol. For purposes of the present invention, though, the term ketal refers to a molecule having the resultant ketal/acetal structure regardless of the reaction used to form the group.

The aromatic moiety may be any substituted or non-substituted (except for the oxo-hetero moiety when present as a substituent rather than a backbone constituent) moiety that includes at least one aromatic ring structure. The aromatic ring structure may be fused or non-fused with respect to other aromatic or aliphatic ring structures (e.g., as when two substituents of any such aromatic ring are co-members of a ring structure). The aromatic moiety optionally may incorporate one or more hetero atoms such as 0, P, S, Si, N and/or the like as constituents and/or substituents of aromatic or aliphatic moieties incorporated into the aromatic moiety.

The linking group that links the aromatic moiety to the alpha, chiral carbon generally desirably may be a single bond or any saturated or unsaturated divalent moiety. The linking group optionally may incorporate one or more hetero atoms such as 0, P, S, N, Si, and/or the like and may be substituted or non-substituted. Preferably, the linking group is a linear, branched or cyclic alkylene radical containing from 1 to 15 carbon atoms, preferably 1 to 5, more preferably 1 to 2 carbon atoms. Most preferably, the linking group is a single bond or an alkylene group of 1 to 6 carbon atoms such as CH₂—.

The optional oxo-hetero moiety refers to a moiety that incorporates a hetero atom bonded to one or more oxygen atoms. Optionally, the oxo-hetero moiety may further include other moieties bonded directly to the hetero atom and/or to an oxygen atom. As used herein, hetero with respect to the oxo-hetero moiety refers to an atom other than carbon and oxygen that has two or more valencies. Examples of such hetero atoms in the context of the oxo-hetero moiety include P, S, Si, N, combinations of these, and the like.

In other contexts of the invention, other hetero atoms may further include O. Of these, S is preferred. Preferred oxo-hetero moieties have the formula

R¹—S(O)(O)—

wherein R¹ may be any monovalent substituent and each O is bonded to the S by a double bond. In representative embodiments, the R¹ moiety may be any linear, branched or cyclic monovalent moiety such as alkyl, aromatic, aralkyl, and the like. The R¹ moiety may be substituted or unsubstituted and may further include one or more hetero atoms such as 0, P, S, Si, and/or N.

In preferred embodiments, the material to be epimerized and resolved includes a mixture of stereoisomers having the following formula:

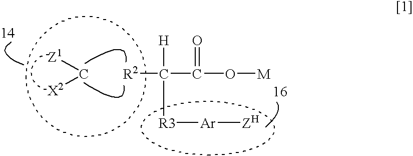

wherein Z¹, Z² (per above, Z¹, Z² may be co-members of a ring structure as shown by the dotted line connecting these two moieties), and M are as defined above; R² designates a trivalent linking moiety that links the ketal moiety to the alpha, chiral carbon; R³ represents a single bond or a divalent linking group; Ar designates a substituted or unsubstituted moiety comprising an aromatic ring; and Z^{H} designates at least one oxo-hetero moiety that is pendant from the aromatic moiety as a substituent thereof.

In preferred embodiments, the ketal moiety and its linking group, shown collectively within the dotted line boundary 14, have the formula

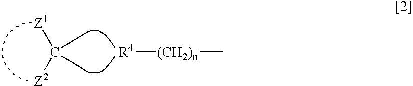

wherein R⁴ together with the C atom of the ketal moiety form a cyclic moiety of 4 to 8, preferably 5 or 6 atoms; and n is 0 to 15, preferably 1 to 6. In preferred embodiments, R⁴ together with the C atom of the ketal moiety form a 5 or 6 membered ring in which all atoms of the ring structure are selected from C, O, S, and N, more preferably from C and O, and most preferably are C atoms. One specific example of a the ketal moiety and its linking group has the formula

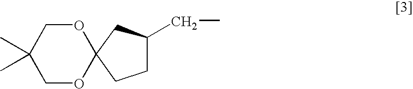

In the preferred embodiments, the aromatic moiety, its linking group, and the oxo-hetero moiety, shown collectively with the dotted line boundary 16, have the formula

—(CH₂)ₘ—Ar-(Z^{H})_{q} [4]

wherein m is 0 to 15, preferably 0 to 6; Ar is the aromatic moiety as defined above; Z^{H} is the oxo-hetero moiety per above; and q is I or more, preferably 1 or 2. In more preferred embodiments, the aromatic moiety, its linking group, and the oxo-hetero moiety, shown collectively with the dotted line boundary 16, have the formula

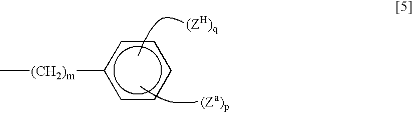

wherein each Z^{H} is as defined above and can be positioned ortho, meta, and/or para relative to the linking group (—CH₂—)ₘ; m is as defined above; each Z^{a} occupy remaining valent sites on the aromatic ring not occupied by a Z^{H} moiety and can be H or any other substituent or a co-member of a ring with another substituent; p is 0 to 4, preferably 4; and q is 1 to 4, preferably 1, with the proviso that p+q is 5. One specific example of an embodiment of the aromatic moiety, its linking group in the form of a single bond, and the oxo-hetero moiety in the form of a sulfone have the formula

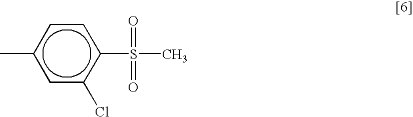

A specific example of an aromatic, ketal acid mixture whose stereoisomers may be epimerized and resolved satisfactorily in the practice of the present invention is the epimer mixture designated schematically as the sulfone 7 in Application B (hereinafter referred to as the aromatic ketal acid intermediate of the compound of Formula I) and which has the formula:

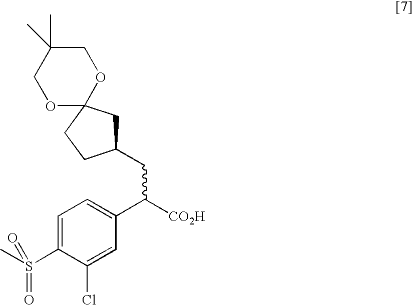

or its salts and derivatives. This structure designates a material including both the 2R,3′R stereoisomer (hereinafter referred to as the R,R stereoisomer) and the 2S,3′R (hereinafter referred to as the S,R stereoisomer) stereoisomers. The R,R stereoisomer is a useful intermediate in the synthesis of the compound of Formula I.

To carry out epimerization, the methodology of the present invention includes the step of subjecting the aromatic ketal acid material to a base-promoted epimerization in a solvent system comprising at least two solvents. For purposes of discussion, the present invention will be explained with respect to a starting material that includes two stereoisomers wherein it is desired to convert the second stereoisomer to the first one via base-promoted epimerization and then optionally to recover the first isomer in purified form via techniques such as crystallization and/or re-crystallization followed by filtering and drying. In the context of the aromatic ketal acid intermediate of the compound of Formula I, this would involve converting at least a portion of the S,R stereoisomer to the desired R,R isomer, for use as an intermediate in a synthesis of the compound of Formula I.

The first solvent used for epimerization is one in which the second stereoisomer is more soluble than the first epimer such that the first epimer preferentially precipitates in the first solvent relative to the second epimer. It is also desirable that the first epimer has some, but a limited, solubility in the first solvent. If the first epimer is too soluble in the first solvent, too little of the undesired, second epimer would be converted to the first, desired epimer. If the first epimer is too insoluble in the first solvent, the epimerization reaction could be too slow. In the meantime, if the second isomer is too insoluble in the first solvent, the reaction may proceed too slowly and/or too little of the second isomer may be converted to the first isomer. Additionally, the conversion of the second epimer to the first epimer tends to increase as the solubility difference between the two epimers in the first solvent increases. Thus, it is desirable that the first epimer have some limited solubility in the first solvent and that the second epimer is not only more soluble, but is as soluble in the first solvent as practically possible. As suggested guidelines in representative modes of practice, a first solvent is suitable when the first epimer desirably has a limited solubility of from about 10 mg/ml to about 150 mg/ml, preferably, from about 10 mg/ml to about 50 mg/ml, more preferably from about 15 mg/ml to about 30 mg/ml, and when the first epimer preferentially precipitates from the solution relative to the second epimer. This solubility desirable is determined at a temperature at which the epimerization is carried out.

The solubility of a first epimer in a solvent may be determined as follows. Sampling syringes and saturated slurry samples are pre-heated (or pre-chilled) to the desired epimerization temperature, e.g., 65 C or 70 C in some embodiments. The syringe is preheated (or pre-chilled) to the same temperature in a vessel containing only the solvent and the syringe. The syringes may be 5 ml disposable syringes. The saturated slurry samples are prepared by combining the solvent under investigation and enough of the purified first epimer to ensure that at least some precipitate is present so that the sample slurry is saturated. A purified epimer refers to an epimer having a purity of at least 85% by weight with respect to the total weight of epimers, preferably at least 90% by weight, more preferably at least 95% by weight. When both the sample and the syringe are at the desired temperature, 1.5 ml samples are quickly drawn into the syringe. The sampling syringes desirably acquire samples through a suitable filter to avoid bringing solids into the syringe. A 25 mm, 0.45 micron PTFE disk filter is suitable. 1 ml of each sample is blown down and then diluted in 10 ml of a solvent in which the epimer is fully soluble such as methanol. HPLC analysis is then performed to determine the amount of epimer in the diluted sample. Three external wt/vol standards were used to determine the wt/vol assay for each sample.

Using this analysis, the R,R epimer has a solubility of 23.24 and 27.05 mg/ml in denatured ethanol (2B-3) at 65 C and 70 C, respectively. Thus, the R,R epimer has the desired limited solubility in this solvent. In contrast, the R, R epimer has a solubility of about 0 mg/ml in heptane at 65 C and 70 C. Thus, the R,R epimer is substantially insoluble in heptane at these temperatures. As used herein, substantially insoluble means that an epimer has a solubility of less than 10 mg/ml, preferably less than 5 mg/ml, and more preferably less than about 0.5 mg/ml in the solvent at the desired epimerization temperature.

In the context of the aromatic ketal acid intermediate of the compound of Formula I, suitable examples of the first solvent would include ethanol, isopropyl alcohol, combinations of these and the like.

The second solvent used for epimerization is one in which the first stereoisomer is substantially insoluble and in which the second isomer has greater solubility. In the context of the aromatic ketal acid intermediate of the compound of Formula I, examples of suitable second solvents include hydrocarbon materials such as heptane.

Generally, a material such as isopropyl alcohol might have the requisite solubility characteristics for use as the first or second solvent in various contexts, but its use at the epimerization stage could lead to undesirable by-products. Accordingly, it is desired that neither the first or second solvent include any isopropyl alcohol during epimerization while the stereoisomers are exposed to basic conditions. As discussed below, however, isopropyl alcohol is beneficially used as a part of a solvent combination after epimerization is complete to carry out a preferential recrystallization that further upgrades the purity of the recovered isomer.

The use of a combination of solvents to carry out epimerization is advantageous. The presence of the first solvent provides a medium in which the epimerization reaction can occur at a reasonable rate. In a representative reaction medium in which the first solvent includes ethanol and the base is ethoxide, the base is believed to deprotonate the chiral carbon atom that is in an alpha position relative to the carboxyl moiety. In practical effect, it is believed that the base removes a hydrogen from this carbon. In the meantime, the ethanol serves as a proton source (e.g., a source of H) to re-protonate the chiral carbon. As this occurs, the less soluble, first isomer preferentially precipitates, driving the equilibrium to preferentially produce even more of the less soluble isomer. Advantageously, the presence of the second solvent such as heptane or another, nonpolar solvent, causes even more of the desired, less soluble first isomer to further precipitate. This extends the system equilibrium even further so that greater amounts of the second isomer are converted to the desired first isomer. In case the second isomer has a limited solubility in the second solvent, the amount of the second solvent added is limited, though, so that enough first solvent remains to keep the second epimer in solution.

In contrast, if the first solvent were to be used on its own, too little of the second isomer might be converted to the desired first isomer. Further, if the second solvent were to be used on its own, the epimerization reaction could occur too slowly to be practical for large-scale production. By using the solvent combination in accordance with the present invention, the advantages of each solvent are realized without experiencing drawbacks associated with their individual uses.

For instance, in a representative context that involves epimerizing the racemate of the aromatic ketal acid intermediate of the compound of Formula I, using only ethanol to carry out epimerization may reach equilibrium when only about 85% of the material is in the R,R form. Attempting to drive the equilibrium farther may tend to produce undue amounts of aryl ethoxy and/or des-chloro impurities. In contrast, when using a mixture of solvents in accordance with the present invention, the equilibrium is driven further so that about 94 to 96% of the material is in the R,R form. Again, attempting to drive the equilibrium farther may tend to produce undue amounts of aryl ethoxy and/or des-chloro impurities. The purity can be further upgraded, though, by dissolving and then preferentially recrystallizing the R, R form in a solvent mixture including a phase that is a nonsolvent for the R, R form. An exemplary recrystallization procedure is described below.

In addition to the first and second solvents, epimerization generally occurs in the presence of one or more other ingredients that include at least one base. The base desirably is one that is sufficiently strong to deprotonate the chiral carbon atom that is in an alpha position relative to the carboxylic acid (or salt) moiety without otherwise unduly degrading other features of the isomers. A wide range of bases would be suitable. Representative examples include an alkoxide such as sodium ethoxide, t-butoxide, sodium isopropoxide, combinations of these, and the like.

Generally, enough base is added, optionally in moderate excess, to help ensure that the reaction to proceeds substantially to equilibrium. Lesser amounts may be used when the stereoisomers may have some base sensitivity, but the yield of the desired isomer may be reduced. Using too much is not necessarily harmful when the stereoisomers are base-tolerant, but using more than is required is less efficient, wastes reagents, and potentially accelerates impurity formation. Balancing these concerns, it is desirable to add enough base so that the base is present in a slight stoichiometric excess, e.g., 1.1 to 1.7, more preferably 1.2 to 1.5 equivalents per equivalent of epimer material.

The base promoted epimerization may be carried out in the two solvents in the presence of the base in a variety of ways. According to one reaction scheme, the stereoisomers are included in an admixture that also includes the first and second solvents and the base. The mixture would be stirred at a suitable temperature for a suitable time period to allow the epimerization reaction to reach its completion given equilibrium constraints.

A particularly preferred epimerization methodology includes carrying out the epimerization reaction in multiple stages. In an initial stage, the epimerization is carried out in a reaction medium that includes the base, the first solvent, and optionally a portion of the second solvent. More desirably, though, no second solvent is included at this stage. This stage allows the initial epimerization to occur at a faster rate than would occur if too much of the second solvent were to be present initially.

After allowing the reaction to proceed in this first stage for a suitable amount of time at a suitable temperature, an additional stage involves gradually adding and increasing the relative amount of the second solvent in the reaction medium. The addition is limited to help ensure that enough first solvent is present to keep the second epimer in solution. The second stage extends the equilibrium further so that more of the second isomer is converted to the desired first isomer. This can be achieved by adding greater amounts of the second solvent so that the volume of the reaction admixture tends to increase over time. It is more efficient and uses less solvent, however, if the relative amount of second solvent is increased using a feed distillation technique in which at least a portion of the first solvent is exchanged with the second solvent. According to this technique, a reaction vessel that includes the stereoisomer material, the first solvent, the desired base, and optionally a portion of the second solvent is distilled to preferentially drive off the first solvent. As the first solvent is driven off, comparable amounts of the second solvent are added so that the volume of the reaction mixture stays generally constant, e.g., the volume ratio of the starting reaction mixture to the volume of the mixture during the course of the reaction is in the range from about 1:5 to 5:1, preferably 1:2 to about 2:1, more preferably about 1:1. The distillation occurs slowly enough so that the second solvent is added over a period long enough to allow the epimerization reaction to proceed at an adequate rate. By way of example, this period may occur over a period ranging from 1 minute to as long as 36 hours. A time period in the range of 30 minutes to 4 hours would be more desired. Optionally, the second stage may be repeated one or more times.

After the second stage(s) of epimerization reaction is/are finished, the crystallized reaction product will be rich in the desired first isomer. For instance, in the case of the aromatic ketal acid intermediate of the compound of Formula I, the product that crystallizes in a two stage reaction involving ethanol as the first solvent, heptane as the second solvent, and ethoxide as the base may include about 94 to about 96 parts by weight of the RR isomer and only about 4 to about 6 parts by weight of the SR isomer.

The admixture at the completion of the epimerization reaction may still include base. Consequently, the reaction optionally can be quenched by adding a suitable acid so that remaining base does not cause the epimerization to go backwards. The acid should be of moderate strength, because, if the acid is too strong, the acid can degrade the ketal moiety. Examples of suitable acids of moderate strength that are reasonably compatible with the ketal group include organic acids such as citric acid, acetic acid, succinic acid, tartaric acid, malonic acid, malic acid, and combinations of these, and the like. Desirably, only enough acid is added to ensure that remaining base is neutralized inasmuch as too much excess acid risks degradation of the ketal group even when using an acid of moderate strength. Too much acid can also lower yield by converting the salt of the product to an acid.

After epimerization, the purified product may be recovered and used as is, but it may be desirable in some instances to subject the reaction product to further processing. For instance, recrystallization techniques may be used to further upgrade the purity of the desired first isomer. Additionally, recrystallization can improve handling or other characteristics of the purified isomer. In the case of the epimerized aromatic ketal acid intermediate of the compound of Formula I, the recovered R, R isomer may only have a purity of about 95% at the completion of epimerization due to factors including equilibrium constraints. Also, the R,R, isomer crystallizes as very fine particles in ethanol or heptane and has very poor filtering characteristics as a consequence. Carrying out an optional preferential recrystalliztion after epimerization not only can upgrade the purity to a point where the recovered R,R isomer has a purity over 99% by weight, but also the filtering characteristics of the product can be dramatically improved as well. Whereas the epimerized, R,R isomer-rich intermediate tends to form very fine crystals in heptane or ethanol, recrystallization in another solvent such as isopropyl alcohol provides material that filters much more easily. Note that isopropyl alcohol is desirably avoided during the epimerization reaction to avoid undesirable by-products, but may be advantageously used during preferential recrystallization. Very little if any chemical reactions are occurring at the recrystallizing stage so that the risk of by products is minimized.

A representative mode of practice will now be described that involves providing a racemate including approximately equal amounts of the R, R and S, R isomers of the aromatic ketal acid intermediate of the compound of Formula I, subjecting this intermediate to epimerization to obtain a purified R, R product, and then recrystallizing the purified R, R isomer to further upgrade its purity and to improve its filtering characteristics. The different solubility characteristics of the R, R and S, R stereoisomers makes them very suitable for use in the practice of the present invention.

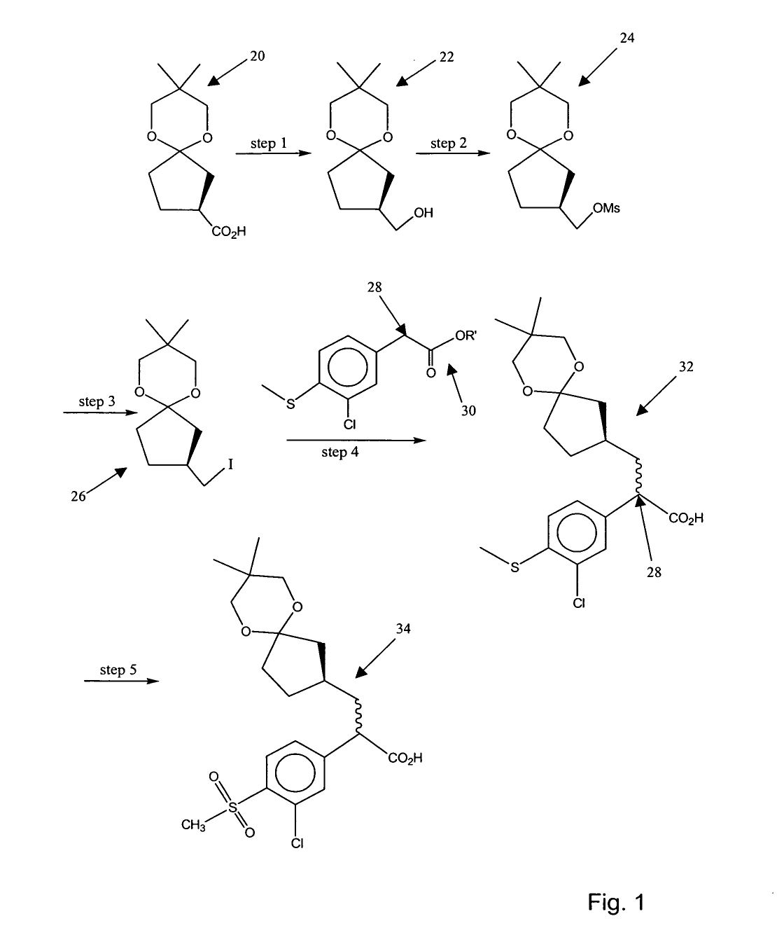

According to this representative mode of practice, a mixture of R, R and S, R stereoisomers according to Formula (7) are provided. The mixture of stereoisomers may be provided from a variety of different sources. One approach involves preparing these stereoisomers according to a synthesis scheme described in Application B. An illustrative synthesis scheme is shown schematically in FIG. 1. In step 1, a chiral, ketal acid 20 is converted via reduction to the corresponding chiral, ketal alcohol 22. The chiral ketal acid may be obtained in an desired fashion such according to procedures described Application A. In step 2, the chiral ketal alcohol 22 is converted to the corresponding chiral mesylate 24, wherein the —OMs moiety has the formula:

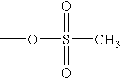

In step 3, the chiral mesylate 24 is converted to the chiral iodide 26. In Step 4, the chiral iodide 26 is used to alkylate the alpha carbon 28 of the substituted aromatic acid ester 30. The aromatic moiety of the ester 30 includes methylthio and chloro substituents. R′ of ester 30 is ethyl. The racemic reaction product 32 includes two stereoisomers that are racemic with respect to the alpha carbon 28. In step 5, the methyl thio group of product 32 is oxidized to form a methyl sulfone substituent. The resultant racemic reaction product 34 constitutes the racemic aromatic ketal acid intermediate of the compound of Formula I to be subjected to epimerization according to the present invention.

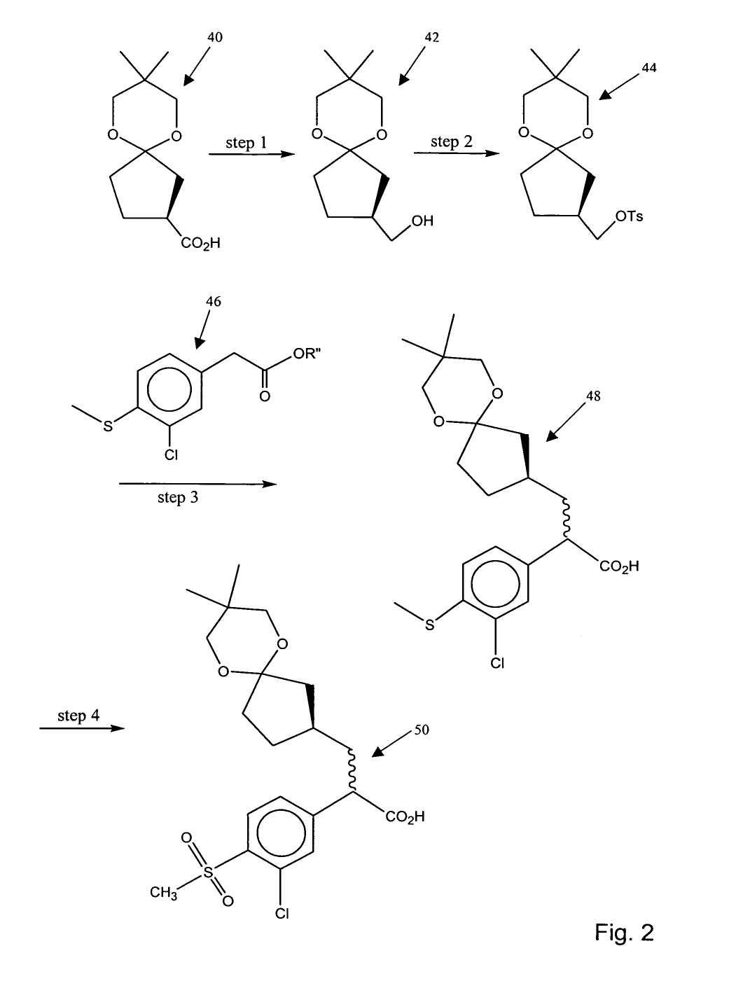

An alternative scheme for providing the racemic aromatic ketal acid intermediate of the compound of Formula I is shown in FIG. 2. The reaction scheme shown in FIG. 2 is described in more detail in the co-pending Application D. As an overview, a ketal acid starting material 40 is reduced to form the alcohol 42 in step 1. This step may be carried out in the same manner as is step 1 in FIG. 1. An alternative manner is included in the examples, below. This alcohol 42 is then converted to the tosylate 44 in step 2, wherein the —OTs moiety has the formula:

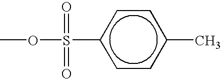

This tosylate 44 is then reacted with the substituted, aromatic ester 46 to yield the racemic reaction product 48 in step 3. The R″ moiety is desirably ethyl. In step 4, the methyl thio substituent of reaction product 48 is oxidized to form a methyl sulfone substituent. The resultant racemic reaction product 50 constitutes a racemic aromatic ketal acid intermediate of the compound of Formula I to be subjected to epimerization according to the present invention. Procedures for carrying out steps 1 though 4 of FIG. 2 are included in the examples, below.

With the racemic aromatic ketal acid intermediate of the compound of Formula I on hand, a first stage of epimerization may be carried out in a first solvent. For purposes of recovering the R, R isomer, a suitable first solvent is ethanol and a convenient base is sodium ethoxide. Examples of other solvents that could be used include IPA, combinations of these, and the like. Other suitable bases include sodium ethoxide, sodium isopropoxide, combinations of these, and the like.

The racemic material, the first solvent and the base are charged to a reaction vessel. Generally, using from about 100 to about 1000 parts by weight of solvent per 100 parts by weight of racemic material would be suitable. Less solvent could be used if desired, although the mixture can be too thick to stir conveniently if too little solvent is present. More solvent can be used, although this would be wasteful. Enough of the base is added to help ensure that the epimerization reaction proceeds all the way to equilibrium at a suitable rate. Generally, using from about 5 to about 50, more preferably from about 10 to about 30 parts by weight of base per 100 parts by weight of racemic material would be suitable.

The first stage of epimerization is carried out for a sufficient period of time at a suitable temperature. A suitable time period may range from 10 minutes to three days, often from about 30 minutes to about ten hours. The reaction medium may be maintained at a wide range of temperatures, although the reaction proceeds at a more reasonable rate when the reaction medium is moderately heated. According, preferred reactions occur at a temperature in a range from room temperature up to the reflux temperature of the reaction medium. When the first solvent is ethanol, a suitable temperature is in the range of 50 C to about 70 C, often about 65 C. The progress of the reaction can be monitored for completion using any suitable technique, such as chiral HPLC, to assess the relative amounts of the R, R and the S, R isomers. Often, it is desirable to cool the mixture, e.g., to about 55 C or less, for sampling. The reaction mixture may be re-heated while waiting for sample results.

After the first stage is complete (e.g., about 84 to about 85 weight percent of the epimers are in the desired epimer form), the second stage of epimerization may be carried out. According to one technique for carrying out the second stage of epimerization, a single charge of the second solvent is added to the reaction medium to drive the equilibrium even further in favor of the R, R monomer. Examples of suitable solvents to use at this stage include heptane, hexane, combinations of these, and the like. In such second solvents, the R, R stereoisomer is generally substantially insoluble, whereas the S, R isomer is more soluble. In representative modes of practice, using from about 100 to about 2000, preferably about 300 to about 1000 parts by weight of the second solvent per 100 parts by weight of the stereoisomers would be suitable. According to another technique for carrying out the second stage of epimerization, the second solvent may be added gradually to the reaction medium, preferably to exchange ethanol being removed by distillation or other suitable technique.

The second stage of epimerization is allowed to proceed for a suitable time period at a suitable temperature. Generally, a suitable time period is in the range of from about 20 minutes to about 48 hours, more desirably from about 30 minutes to about 8 hours. The second stage of epimerization may be carried out at the same or at a different temperature as the first stage. Desirably, temperatures in the range of about 25 C up to the reflux temperature of the reaction medium would be suitable, although the reaction proceeds faster at warmer temperatures. Often, it is convenient to maintain the same temperature during the second stage as was maintained in the first stage. The reaction mixture may be monitored to assess when epimerization is completed.

After epimerization is completed, the reaction mixture is cooled, e.g., to a temperature in the range of 0 C to about 30 C, more desirably 15 C to 25 C, and the base desirably is at least partially neutralized with an acid to quench the reaction. In one scheme, quenching is achieved using acetic acid or the like. This quenching acid may be charged directly or added via a solution of the acid in suitable solvent such as methanol.

Preferably, subsequent dissolution and recrystallization steps are carried out to further upgrade the purity of the desired R, R isomer and to improve filtering characteristics. To accomplish this, enough dissolution solvent is added in order to dissolve the reaction product. This can be added before, after or during removal of the remaining first and second solvents, but any remaining first and second solvents desirably are removed before proceeding to the optional recrystallization step. Methanol is one example of a suitable dissolution solvent inasmuch as both the R,R and S,R isomers are very soluble in methanol.

To achieve recrystallization, a suitable solvent that preferentially is a nonsolvent for the R, R stereoisomer and a good solvent for the S, R stereoisomer is added either in a single charge or gradually to the mixture containing the methanol and the reaction product. Isopropyl alcohol was found to be a suitable nonsolvent in which the R,R stereoisomer preferentially recrystallizes relative to the S,R stereoisomer. The dissolution solvent, e.g., methanol, optionally may be removed as the nonsolvent is added in order to maintain a substantially constant volume. The recrystallization mixture desirably is moderately heated and stirred during recrystallization. A suitable temperate may be in the range of from about 25 C to about 65 C, often about 50 C. After a desired period of time, e.g., 10 minutes to 24 hours, the recrystallization medium is slowly cooled and aged at the cooled temperature. Cooling may occur over a period from 5 minutes to 8 hours, typically from about several seconds to 3 hours. The mixture may be cooled to a temperature in the range of from about 0 C to about 25 C, desirably 10 C to about 20 C. Aging may then occur at a cooled temperature for a suitable time. In some modes of practice, aging for a period of from about 10 minutes to two days, desirably 30 minutes to 4 hours, would be suitable.

After the recrystallization, the reaction mixture may be filtered to collect the precipitated product. The product may then be dried under suitable conditions. By way of example, drying may occur for 10 minutes to 36 hours at a reduced pressure and at a temperature in the range of from about 35 C to about 55 C.

The present invention will now be described with reference to the following illustrative examples.

### Example 1

### Synthesis of Tosylate According to Steps 1 and 2 in FIG. 2

### Salt Cleavage and (S)-Ketal-Acid Concentration

A 12,000 L glass-lined vessel was charged with 252.4 kg (752.4 mol) of (S)-Ketal-acid, (S)-MBA salt precursor of acid 20 of FIG. 1, followed by 1260 L (liters) toluene. The mixture (slurry) was cooled to 5° C. under nitrogen with agitation. To a 16,000 L glass-lined vessel was charged 212 L potable water followed by 318.0 kg 50% aqueous citric acid. The aqueous citric acid solution was cooled to 0° C. with agitation and then added to the ketal-acid salt slurry over 20 min while keeping the temperature of the reaction mixture below 5° C. The two-phase reaction mixture was warmed to 13° C. and allowed to settle for 30 min. The lower aqueous layer was separated. To the aqueous citric acid layer was added 504 L toluene. The two-phase mixture was stirred for 15 min at 14° C. and allowed to settle for 49 min at 14° C. The lower aqueous layer was separated. The two toluene extracts containing the intermediate (S)-ketal acid were combined and 84 L potable water was added. The two phase mixture was stirred for 17 min at 16° C. and the mixture allowed to settle for 60 min at 16° C. The lower aqueous layer was separated into a separate vessel.

To this aqueous solution was added 504 L toluene. The two-phase mixture was stirred for 20 min at 18° C. and allowed to settle for 30 min at 18° C. The lower aqueous layer was separated and combined with the aqueous citric acid solution and discarded. All of the toluene phases containing the (S)-ketal acid were combined and approximately 1,018 L of the toluene solution of the (S)-ketal-acid was transferred from the 12,000 L glass-lined vessel to a 2000 L Hastelloy vessel. Transfer of 1018 L of solution to the 2,000 L Hastelloy vessel provided for a significant amount of head space for the subsequent distillations to minimize the chance of bumping the batch into the vessel overheads.

The solution was concentrated via a feed-distillation under reduced pressure (30-40 mm pressure, vessel temperature ˜35° C. with a maximum bath temperature of 50° C.) until the volume of the (S)-ketal-acid solution reached 588 L. After the 12,000 L feed vessel is empty, the distillation was halted and the feed vessel rinsed with 84 L toluene to the distillation vessel. The distillation was then restarted and continued until the target volume was reached. The solution was sampled for Karl Fischer analysis and showed 0.007% contained water. The solution of the ketal-acid was then cooled to 10° C. prior to the feed to a Vitride solution (Rohm & Haas). Vitride is an aluminumhydride reducing agent. The full chemical name of Vitride is Sodium Dihydro-bis-(2-Methoxyethoxy) Aluminate or SDMA. It is highly soluble in aromatic hydrocarbon solvents and is sold as a 70% solution in toluene.

### (S)-Ketal-Acid Reduction

A 2,000 L glass-lined vessel was charged with 487.8 kg 70% Vitride solution in toluene followed by 441 L toluene with agitation. Approximately 9 L of toluene is used to flush out the charging dip leg after the Vitride charge. After the toluene charge, a recirculation loop containing a ReactIR™ monitoring instrument was started to monitor the reduction. The diluted Vitride solution was cooled to <5° C. The pre-cooled ketal-acid solution was transferred to the Vitride solution through a 20-micron polishing filter and ¼″ mass-flow meter at a rate of 2.0 kg/min. A mass flow meter was utilized as a safety precaution to minimize the risk of adding the ketal-acid at a rate that would generate hydrogen faster than could be safely handled in the reduction vessel. The reaction is very exothermic but the heat and hydrogen flow is completely controlled by the ketal-acid feed rate. A maximum addition rate was 2.2 kg/min. A polishing filter was used to prevent any residual salts from plugging the relatively small mass flow meter. A total of 581 kg of ketal-acid solution was transferred (density 0.959 kg/L)

The reaction temperature was maintained at <25° C. but with a target range of 20±5° C. throughout the ketal-acid addition. Running the reduction at a lower temperature (e.g. <10° C.) results in lower yields, presumably due to incomplete reduction. Maintaining ambient temperature for the reaction results in higher yields.

The vessel containing the ketal-acid solution was rinsed with 42 L toluene and the rinse transferred through the filter and mass-flow meter. The reduction reaction mixture was agitated for 70 min at 20-22° C. and sampled for reaction completion. The reaction was monitored by the ReactIR™ to check for the presence of the excess Vitride at the end of the reaction, but an HPLC sample was also taken to check for the presence of unreacted ketal-acid. To a 12,000 L glass-lined vessel was charged 596.8 kg 20% aqueous NaOH solution which was cooled to 2° C. with agitation. This quantity of 20% NaOH used for this batch (500 L, 600 kg) was determined by the minimum stirrable volume of the 12,000 L vessel used for the quench. The amount of NaOH can be reduced where practical concerns like this do not control. The recirculation loop used for the ReactIR™ was blown back into the reactor just prior to the quench.

The reaction mixture was then transferred to the aqueous NaOH solution through a ½ mass flow meter while keeping the temperature of the quench mixture below 25° C. A maximum feed rate was set at 9 kg/min to control the hydrogen evolution. The addition time for this batch was 3 h with a maximum temperature of 16° C. (1,461 kg of reaction solution transferred).

The reduction reaction vessel was rinsed with 84 L toluene and the rinse transferred through the mass flow meter. The quench mixture was warmed to 16° C. and stirred for 1 h at 16-17° C. The agitation was stopped and the two-phase mixture allowed to settle for 1 h at 17° C. The lower aqueous layer containing the caustic aluminum salts was separated into another glass-lined vessel. To this aqueous solution was added 504 L toluene and the two-phase mixture stirred for 30 min at 21° C. and allowed to settle for 1 h at 21-22° C. The layers were separated and the two toluene layers containing the crude ketal-alcohol were combined followed by a 84 L toluene vessel rinse. To the aqueous layer was added 504 L toluene and the two-phase mixture stirred for 30 min at 18° C. and allowed to settle for 1 h at 18° C. The lower aqueous phase was separated and discarded (638 L for this batch).

The two toluene layers containing the crude ketal-alcohol were again combined followed by a 84 L toluene vessel rinse. To the total solution containing the intermediate ketal-alcohol was added 209 L potable water. The two-phase mixture was stirred for 38 min at 18-21° C. and allowed to settle for 1 h at 21° C. The water rinse serves to remove any residual salts, but also removes some of the 2-methoxyethanol liberated during the quench as well as the intermediate ketal-alcohol thus requiring toluene back-extractions to minimize yield loss. The lower aqueous phase was separated and to it was added 211 L toluene. The two-phase mixture was stirred for 30 min at 24° C. and allowed to settle for 1 h at 24° C. The toluene layer was recombined with the bulk ketal-alcohol solution followed by a 84 L toluene vessel rinse. To the aqueous layer was added 210 L toluene. The two-phase mixture was stirred for 40 min at 23° C. and allowed to settle for 1.7 h at 23° C. The layers were separated and the aqueous layer discarded (399 L for this batch). The toluene layer was recombined with the bulk ketal-alcohol solution followed by a 84 L toluene vessel rinse. At this point, the ketal-alcohol solution was sampled for 2-methoxyethanol which was then monitored during the subsequent feed distillation. Approximately 1,018 L of the toluene solution of the ketal-alcohol was transferred from the 12,000 L glass-lined vessel to a 2000 L Hastelloy vessel. The solution was concentrated via a feed-distillation under reduced pressure (20 mm minimum pressure, vessel temperature ˜30-35° C. with a maximum bath temperature of 50° C.) until the volume of the ketal-alcohol reached 320 L. The ketal-alcohol solution was held for ˜1 h and any second-phase water present removed prior to starting the feed distillation.

After the initial feed distillation was complete, the ketal-alcohol solution was sampled for 2-methoxyethanol and water content. It was necessary to add additional toluene and continue the distillation to remove the 2-methoxyethanol to an acceptable level. A total of three additional toluene charges were required (100 L, 150 L and 500 L) with the final distillation volume being reduced to 220 L. The final 2-methoxyethanol content was 0.022% relative to the ketal-alcohol. Since the ketal-alcohol solution was to be eventually transferred back to the 12,000 L vessel for the tosylation reaction, no vessel rinse was performed during the distillation.

### Tosylation

The toluene solution of the intermediate ketal-alcohol was transferred to a 12,000 L glass-lined reactor followed by a 150 L toluene rinse. The 2,000 L Hastelloy reactor was vacuum dried and to it was charged 105.9 kg (944.0 mol) 1,4-diazabicyclo[2.2.2]octane (DABCO) followed by 605 L toluene. The mixture was stirred for 1.2 h at 15-16° C. until the solids were dissolved. The DABCO solution was combined with the solution of the ketal-alcohol followed by a 42 L toluene vessel rinse. The vessel used for the DABCO solution make-up was again vacuum dried and to it charged 162.1 kg (850.2 mol) p-toluene sulfonyl chloride (tosyl chloride) followed by 542 L toluene. The mixture was stirred for 15 min at 10-16° C. to dissolve the solids (dissolution is endothermic) and then cooled to 2° C. The solution of tosyl chloride was then transferred to the solution of the ketal-alcohol and DABCO while keeping the reaction temperature <10° C. (addition performed over ˜3 h with a temperature range of −2 to +6° C.). To the vessel containing the tosyl chloride was added 43 L toluene as a vessel rinse. The reaction was stirred for 1 h at 3 to 4° C. and sampled for reaction completion (HPLC). The reaction completion showed 1 mg/mL ketal-alcohol remaining with excess tosyl chloride still present.

While the reaction completion sample was been analyzed, a 16,000 L glass-lined vessel was charged with 700 L potable water followed by 63.8 kg (759 mol) sodium bicarbonate. The mixture was stirred at ambient temperature to dissolve the solids. Once the tosylation reaction was deemed complete, the reaction mixture was added to the aqueous bicarbonate solution over ˜2 h at ambient temperature (jacket temperature setpoint of 20° C.) followed by 85 L toluene as a vessel rinse. The two-phase mixture was stirred for 2.3 h at 25-28° C. to hydrolyze the excess tosyl chloride and sampled for reaction completion (tosyl chloride not detected). The mixture was allowed to settle for 1 h at 29° C. and the lower aqueous layer separated. To the upper toluene layer was added 504 L potable water. The two-phase mixture was stirred for 30 min at 27° C. and allowed to settle for 1 h at 27° C. The lower aqueous layer was separated, combined with the sodium bicarbonate extract and discarded. The toluene solution of the Step 6 product was filtered through a 10-inch, 20-micron filter to a 12,000 L glass-lined reactor followed by 127 L toluene used as a vessel rinse. The filtered toluene solution was allowed to settle for at least 30 min followed by removal of any second phase water that was present before starting the subsequent feed distillation.

### Crystallization & Isolation

Approximately 1,018 L of the toluene solution of the (S)-chiral tosylate was transferred to a 2,000 L Hastelloy vessel. The solution was concentrated via a feed-distillation under reduced pressure (20 mm minimum pressure, vessel temperature ˜30-35° C. with a maximum bath temperature of 50° C.) until the volume of the (S)-chiral tosylate solution reached 353 L (no toluene rinse of the 12,000 L vessel was performed). The final strip volume prior to the heptane addition is important inasmuch as too much toluene will result in a lower product yield due to losses to the mother liquors.

Approximately half of the product solution was transferred to a 2,000 L glass-lined vessel (180 kg, density 1.07 kg/L) with the other half transferred back to the 12,000 L glass-lined feed vessel for storage. The 2,000 L glass-lined vessel (used as the Heinkel feed vessel) was too small to accommodate crystallizing the entire batch. It was therefore split and the crystallization performed in two parts. The batch was transferred through a mass flow meter to accurately determine how much of the batch was contained in each part. In this case, approximately 220 kg was contained in the second part necessitating that additional heptane above the standard charge be added for the second part crystallization.

To the Hastelloy distillation vessel was added two separate portions of 15 L toluene as a line rinse to each of the two glass-lined vessels. To the 2,000 L glass-lined vessel containing the first half of the batch was added 713.5 kg n-heptane at ambient temperature (20±5° C.) to crystallize the product. To each drum of n-heptane was added 8-10 drops of Octastat 5000 to increase the solvent conductivity. The charge rate of the n-heptane was limited to ≦8 kg/min. The product crystallization occurs during the heptane addition.

After the heptane addition and the product crystallization had occurred, the product slurry was cooled to −15° C. over 5.5 h and allowed to stir for 1 h at −15° C. The cool-down rate after the crystallization is not critical since the batch crystallizes during the heptane addition at ambient temperature. The product was isolated using a Heinkel and washed with pre-cooled n-heptane (<−10° C.) to give 108.1 kg product wet cake. As necessary, the mother liquors were used to rinse product from the crystallization vessel after the initial filtration sequence, especially for the second half isolation. After isolation was completed, the wet cake was loaded to a double-cone dryer and drying initiated (35° C. maximum bath temperature, full vacuum) while the second half of the batch was crystallized.

The second half of the batch was transferred from the 12,000 L glass-lined vessel to the 2,000 L glass-lined vessel used for the first half crystallization followed by 81.1 kg n-heptane as a vessel and line rinse. To the product solution was added an additional 790.6 kg n-heptane at 20±5° C. to crystallize the second half of the batch. After the heptane addition and the product crystallization had occurred, the product slurry was cooled to −15° C. over 10 h and allowed to stir for 1 h at −15° C. to −18° C. The product was isolated using a Heinkel and washed with pre-cooled n-heptane (<−10° C.) to give 140.2 kg product wet cake (248.3 kg total). After isolation was completed, the wet cake was loaded to the double-cone dryer containing the first half of the batch and drying restarted (35° C. maximum bath temperature, full vacuum). The product was dried until an LOD of ≦1.0% was achieved (0.00% LOD obtained on batch, LIMS 38-478512). The product was discharged from the dryer into double poly-lined fiber packs to give 224.1 kg (632.2 mol, 84.0% yield) of the (S)-chiral tosylate. A stirred sample of the mother liquors (˜3,100 L) was obtained which showed that ˜17.5 kg (49.5 mol, 6.6% yield based on 5.7 g/L assay of mother liquor sample) product was contained in the mother liquors.

### Example 2

### Reaction Corresponding to Steps 3 and 4 of FIG. 2

A mixture of 268 g THF and 177.7 g (1.00 equiv) of ethyl (3-chloro-4-(methylthio)phenylacetate were slowly added to a cold (<−15° C.) 20% solution of potassium tert-butoxide in THF (415.5 g, 1.02 equiv) and allowed to react over 2 hours at −15° C. to form a potassium enolate. A 1:1 solution mixture of 256.1 g (1.00 equiv) of the (S)-(8,8-dimethyl-6,10-dioxaspiro[4.5]decan-2-yl)methyl 4methylbenzenesulfonate and 321 g THF was transferred slowly to the cold enolate reaction mixture solution. The alkylation reaction mixture was stirred at <0° C., warmed to 40° C. and then held at 40° C. until the reaction was complete. The THF was distilled off under vacuum and the resulting ester product extracted into 629 g of MTBE and 445 mL water. The bottom aqueous layer was extracted with another 100 g MTBE. The resulting two MTBE/product layers were combined.

The resulting intermediate alkylation product ester as a MTBE solution was directly utilized for a hydrolysis reaction by adding 69 g (1.2 equiv) of 50% aqueous sodium hydroxide. The mixture was heated to 50° C. and stirred until the reaction was complete. The MTBE was removed by distillation under vacuum. The product mixture was extracted into water by adding 613 g water and 612 g toluene. The bottom aqueous product layer was extracted again by adding 612 g toluene to remove any remaining by-products. The bottom aqueous product layer was pH adjusted using 35 g of a 50% citric acid solution to a pH of 8.5-9.0. The aqueous product layer was concentrated under vacuum to remove all residual toluene. This water mixture was carried into the subsequent oxidation reaction.

A solution of 4.8 g (0.02 equiv) sodium tungstate dihydrate catalyst in 12 g water was added to the reaction mixture. The reaction mixture was cooled to <15° C. and 209 g (1.2 equiv) of 30% aqueous hydrogen peroxide was added, maintaining the reaction mixture below 45° C. The mixture was pH adjusted to approximately 7.5 by adding small amounts (˜1-5 g) of a saturated aqueous sodium bicarbonate solution, if necessary. The reaction was stirred at 20° C. until the reaction was complete. The peroxide mixture was quenched using an aqueous solution of sodium sulfite. The quenched reaction mixture was then extracted by adding 407 g isopropyl acetate. The top organic layer was separated to remove by-products. The bottom aqueous product layer was pH adjusted to 4.7 using 140 g of a 50% solution citric acid and extracted by adding 610 g isopropyl acetate. The bottom layer was separated and extracted again with 558 g isopropyl acetate. The combined organic/product layers were distilled under vacuum to remove residual water. The resulting product slurry was heated to 65° C. with an additional 370 g of isopropyl acetate. The mixture was crystallized by slowly adding 498 g n-heptane. The slurry was concentrated under vacuum and an additional 632 g n-heptane added. The mixture was concentrated and slowly cooled to 10° C., aged, filtered, washed with n-heptane and dried to produce 229.7 g (73.8% yield) of (R)-2-(3-chloro-4-(methylsulfonyl)phenyl)-3-(8,8-dimethyl-6,10-dioxaspiro[4.5]-decan-2-yl)propanoic acid as a dry solid powder.

### Example 3

### Epimerization Reaction, Dissolution, Recrystallization, Filtering and Drying

### Sodium Salt Formation

A 2000 L glass-lined reactor (vessel 1) was charged with 99.8 kg (232 mol, 1.00 equiv) of the reaction product 48 shown in FIG. 2 followed by 165.5 kg of denatured ethanol, 2B-3. The mixture was stirred at 20° C. for 10 min. A solution of 112.5 kg 21% sodium ethoxide in ethanol was charged to vessel 1 followed by a line rinse of 5.1 kg ethanol, 2B-3.

### Chiral Epimerization

The mixture was heated to 65° C. and stirred for 6 hours. The mixture was then cooled to 55° C. and sampled by chiral HPLC to determine the diastereomer ratio. After the age with sodium ethoxide in ethanol the percentage of the undesired isomer was expected to be <20% (2S,3′R) relative to the (2R,3′R) isomer by chiral HPLC analysis and, indeed, in the lab, 14.7% to 18.5% (2S,3′R) was observed. This is as far as the epimerization can be taken in pure ethanol without significant production of aryl ethoxy and des-chloro impurities. While waiting for sample results, the mixture was heated back to 65° C.

While maintaining the vessel contents at 65° C., 573.1 kg of heptane was charged and the mixture stirred and aged for 2 h at 65° C. To add the heptane, ethanol is exchanged via a vacuum feed-strip with heptane and the reaction mixture aged for a minimum of a 2 hours. The mixture was then cooled to 55° C. and sample. After the addition and age with n-heptane, the ratio of the (2R,3′R) to (2S,3′R) isomers was monitored along with the production of aryl ethoxy and des-chloro impurities using the chiral HPLC method. The bath temperature was set at 75° C. (the mixture refluxes at approximately 67° C.) and the reaction mixture was concentrated by atmospheric distillation to ˜450 L. The mixture was then cooled to 55° C. and sampled for final epimerization completion. After the distillation of n-heptane/ethanol, the percentage of the undesired isomer was expected to be <8% (2S,3′R) relative to the (2R,3′R) isomer by chiral HPLC analysis, and indeed 5.5 to 7.5% was observed. This is as far as the epimerization can be taken without significant production of aryl ethoxy and des-chloro impurities.

The mixture was cooled to 20° C. and 7.0 kg (117 mol, 0.5 equiv) of acetic acid was charged to a separate vessel 2 followed by a line rinse of 2.0 L methanol. To vessel 2 was then charged 250.0 L methanol and the mixture stirred for 15 min. The mixture in vessel 2 was then transferred to vessel 1 while maintaining the vessel 1 contents at 20 (±5)° C. The reaction mixture was stirred for 15 min at 18° C. The mixture was then sampled to ensure the pH was between 6 and 8. The actual pH was 7.5. To vessel 2 was next charged 750 L methanol which was heated to 40° C. The reaction mixture in vessel 1 was atmospherically distilled while continuously charging methanol from vessel 2 through a ¼″ mass flow meter to maintain a constant volume in vessel 1. The mixture refluxes at about 57° C. About 880 L of distillate is collected. To vessel 2 was then charged 479.8 kg isopropyl alcohol which was heated to 50° C. The reaction mixture in vessel 1 was atmospherically distilled while continuously charging isopropyl alcohol from vessel 2 through a ¼″ mass flow meter to maintain a constant volume in vessel 1. The mixture will begin to reflux at ˜57° C. and this will increase to ˜71° C. The resulting slurry was slowly cooled over 2 h to 20° C. followed by aging at 20° C. for 1 h. The slurry was then sampled for final recrystallization completion. The slurry sample was filtered and the mother liquors analyzed for ratio of (2R,3′R) to (2S,3′R) isomers by chiral HPLC for comparison to reference batches (26.3 to 32.0% area norm (2R,3′R) was observed) to ensure the correct volume had been reached to obtain a good yield of the product. The result was 32% area norm for the (2R,3′R) isomer and 68% area norm for the (2S,3′R) isomer by chiral HPLC analysis, which was consistent with reference batches.

### Isolation and Drying

To a Heinkel rinse vessel (vessel 3) was charged 272.9 kg isopropyl alcohol. The product slurry was filtered through the Heinkel centrifuge filter unit. Each spin was rinsed with a small quantity of the isopropyl alcohol from vessel 3. Each spin was initially filled with 3-5 kg of slurry and the product washed with ˜1 kg of isopropyl alcohol which resulted in about 1.5-2.0 kg of wet cake per spin. The combined isolated wet cakes were transferred to Krauss-Maffei conical dryer and dried under vacuum for ˜18 h at 40-45° C. to produce 94.7 kg (209 mol, 90.3% yield) of (2R,3′R)-sulfone ketal acid, sodium salt [(2R,3′R)-9)] isolated as a dry powder. The isolated material showed excellent purity by chiral HPLC and area normalized purity of 98.91%. However, the assay purity was only 93.6 wt % due to the presence of residual sodium acetate produced during neutralization with acetic acid.

Other embodiments of this invention will be apparent to those skilled in the art upon consideration of this specification or from practice of the invention disclosed herein. Various omissions, modifications, and changes to the principles and embodiments described herein may be made by one skilled in the art without departing from the true scope and spirit of the invention which is indicated by the following claims.

1. A method of epimerization, comprising the steps of:
providing a mixture comprising first and second epimers;
epimerizing the mixture to convert at least a portion of the second isomer to the first epimer, said epimerizing providing an epimerized product with an enriched content of the first epimer and said epimerizing occurring in a basic reaction medium comprising first and second solvents; wherein:
the second epimer is substantially fully soluble in the first and second solvents, and
the first epimer is moderately soluble in the first solvent and substantially insoluble in the second solvent.

2. The method of claim 1, wherein each of the epimers is an aromatic, ketal acid.

3. The method of claim 1, wherein the epimers each are an aromatic ketal acid that comprises:
a carboxylic acid moiety or salt thereof;
a chiral carbon atom that includes an H substituent and that is in an alpha position relative to the carboxylic acid moiety or salt thereof;
a ketal moiety linked to the chiral carbon atom by a linking group;
a substituted aromatic moiety linked to the chiral carbon atom by a single bond or a linking group; and
an oxo-hetero moiety incorporated into the aromatic moiety.

4. The method of claim 3, wherein the epimers respectively have the formula 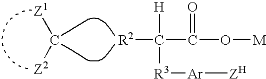
wherein Z¹, Z² (per above, Z¹, Z² may be co-members of a ring structure as shown by the dotted line connecting these two moieties), and M are as defined above; R² designates a trivalent linking moiety linking the ketal moiety to the alpha, chiral carbon; R³ represents a single bond or a divalent linking group; Ar designates a substituted or unsubstituted aromatic moiety; and Z^{H} designates at least one oxo-hetero moiety that is pendant from the aromatic moiety as a substituent thereof.

5. The method of claim 4 wherein the ketal-containing moiety linked to the carbon that is in an alpha position relative to the carboxyl moiety has a structure according to Formulae 2 or 3.

6. The method of claim 4, wherein the —R³—Ar-Z^{H} moiety has a structure according to Formulae 4, 5 or 6.

7. The method of claim 4, wherein the epimers have respective structures according to Formula 7.

8. The method of claim 1 wherein the base comprises an alkoxide.

9. The method of claim 8, wherein the alkoxide comprises an ethoxide.

10. The method of claim 1, wherein the first solvent comprises ethanol.

11. The method of claim 1, wherein the second solvent comprises a hydrocarbon.

12. The method of claim 11, wherein the hydrocarbon comprises heptane.

13. The method of claim 1, wherein the first solvent comprises ethanol, the second solvent comprises heptane, and the base comprises an ethoxide.

14. The method of claim 1, wherein the epimerizing step comprises increasing the amount of the second solvent relative to the first solvent as the epimerization progresses.

15. The method of claim 1, wherein the epimerizing step comprises
a first stage that comprises epimerizing the epimer mixture in a reaction medium comprising the base, the first solvent, and optionally a portion of the second solvent; and
a second stage that comprises epimerizing the epimer mixture in a reaction medium comprising the base, at least a portion of the first solvent, and at least a portion of the second solvent.

16. The method of claim 15, wherein the second stage comprises increasing the amount of the second solvent relative to the first solvent as the epimerization progresses.

17. The method of claim 1, further comprising the step of recrystallizing the epimerization product.

18. The method of claim 17, wherein the recrystallizing increases the purity of the first epimer.

19. The method of claim 7, wherein the first solvent comprises ethanol, the second solvent comprises heptane, and the base comprises an ethoxide.

20. The method of claim 17, wherein the recrystallizing comprises the step of recrystallizing in a solvent mixture comprising first and second recrystallizing solvents, wherein:
the first epimer is substantially soluble in the first recrystallizing solvent and is substantially insoluble in the second recrystallizing solvent; and
the second epimer is substantially soluble in the first and second recrystallizing solvents.

21. A method of epimerization, comprising the steps of:
providing a mixture comprising first and second epimers;
epimerizing the mixture to convert at least a portion of the second isomer to the first epimer, said epimerizing providing an epimerized product with an enriched content of the first epimer and said epimerizing occurring in a basic reaction medium comprising first and second solvents; wherein:
the second epimer is more soluble in the first and second solvents than is the first epimer, and
the first epimer substantially insoluble in the second solvent.
